(19) United States
(12) Patent Application Publication
(10) Pub. No.: US 2019/0027254 A1
(43) Pub. Date: Jan. 24, 2019

(54) MEDICAL INFORMATION PROVIDING APPARATUS, OPERATION METHOD OF MEDICAL INFORMATION PROVIDING APPARATUS, AND MEDICAL INFORMATION PROVIDING PROGRAM

(71) Applicant: FUJIFILM Corporation, Tokyo (JP)

(72) Inventors: Yuanzhong LI, Tokyo (JP); Mizuki TAKEI, Tokyo (JP)

(73) Assignee: FUJIFILM Corporation, Tokyo (JP)

(21) Appl. No.: 16/036,123

(22) Filed: Jul. 16, 2018

(30) Foreign Application Priority Data
Jul. 19, 2017 (JP) 2017-140291

(51) Int. Cl. G16H 50/70; G16H 10/60; G16H 50/20; G16H 50/30; G16H 15/00; G16H 30/20

(52) U.S. Cl. CPC G16H 50/70; G16H 10/60; G16H 50/20; G16H 40/63; G16H 15/00; G16H 30/20; G16H 50/30

(57) ABSTRACT

Finding acquisition means acquires finding data including disease information regarding a brain disease of a target patient and diseased part information regarding a diseased part where the brain disease has occurred. Similar case search means searches for case data similar to the finding data of the target patient as similar case data. Information output means acquires an appropriate range of vital sign data from the similar case data, and outputs information regarding the goal of vital sign data of the target patient.

### CROSS-REFERENCE TO RELATED APPLICATION

This application claims priority from Japanese Patent Application No. 2017-140291, filed on Jul. 19, 2017, the disclosure of which is incorporated by reference herein in its entirety.

### BACKGROUND

### Field of the Invention

The present invention relates to an apparatus for analyzing finding data for a patient and presenting information regarding an appropriate treatment policy for the patient from past cases.

### Related Art

In hospitals, patients with severe symptoms always have a risk of sudden worsening of the condition. These patients have been strictly managed under special circumstances, such as an intensive care room. In a case where a patient is brought in, a biological information monitor is attached, so that vital signs, such as an electrocardiogram, a heart rate, a blood pressure, and a body temperature, are monitored over a long period of time. In recent years, vital signs obtained from biological information monitors are displayed on the screen, and sampling results are recorded in electronic medical charts as electronic data.

In a stroke represented by cerebral hemorrhage and cerebral infarction, blood pressure management after the onset of the disease is considered to be particularly important. In a patient with cerebral hemorrhage, high blood pressure is a risk factor for rebleeding. Therefore, a treatment to lower the blood pressure is performed using an antihypertensive agent or the like. For patients with cerebral infarction, a treatment to promote re-opening of the blood flow is performed by administering a medicine that dissolves the thrombus causing infarction. However, in the case of a hypertensive patient, there is a risk of causing cerebral hemorrhage as a side effect of drug administration. Therefore, it is also important to manage blood pressure.

In such diseases, management in the course of treatment is important. For this reason, a system has come to be provided that quantitatively calculates and displays the degree of occurrence of a change in a patient's condition from the past patients data and predicts a change in the patient's condition. For example, in JP2008-176473A, by using a function for calculating the degree of occurrence of a condition change with a plurality of condition change factors causing a change in a patient's condition as parameters, the degree of occurrence of a change in the patient's condition is understood.

On the other hand, with the introduction of an electronic medical record system, information of a large amount of electronic medical record is accumulated, and support of evidence-based medicine (EBM) is being studied based on the accumulated information. In addition, a wide-area electronic medical record system allowing the exchange of data between medical institutions for the purpose of cooperation in disease diagnosis and sharing of medical information in the community has been realized. As elements forming the wide-area electronic medical record system, there is a picture archiving and communication system (PACS) in addition to the electronic medical record system provided in each medical institution. In the PACS, various kinds of image data including image data received from a modality, such as a computed tomography (CT) apparatus or a magnetic resonance imaging (MRI) apparatus, can be managed in a unified manner using a digital imaging and communication in medicine (DICOM) standard. In particular, in brain diseases, in addition to management of vital signs, treatment based on image examination is important. For this reason, utilization of an electronic medical record system and a medical image management system is desired.

For blood pressure management in brain diseases, in the guidelines issued by each medical system conference, a certain reference value for the allowable blood pressure range is defined. However, since the true allowable range depends on the characteristics of each patient, there are also health care professionals who think that it is difficult to perform optimal treatment for each patient only with the uniformly defined standard values of the guidelines. For example, lowering blood pressure generally reduces the risk of cerebral hemorrhage, but lowering the blood pressure of a person who originally has high blood pressure has a risk that the brain will become ischemic.

In managing blood pressure, information of image examination is also important. For example, in a cerebral hemorrhage patient, the risk of rebleeding changes depending on the position or the size of a hematoma and the presence or absence of an increase tendency. Therefore, it is necessary to perform blood pressure management corresponding to the magnitude of the risk.

### SUMMARY

Therefore, in order to solve the aforementioned problems, it is an object of the invention to provide a medical information providing apparatus for outputting information regarding a treatment policy suitable for a patient based on the analysis result of data, such as an electronic medical record, an operation method of a medical information providing apparatus, and a medical information providing program.

A medical information providing apparatus of the invention comprises: finding acquisition means for acquiring finding data including disease information regarding a brain disease of a target patient and diseased part information regarding a diseased part where the brain disease has occurred; similar case search means for searching for case data having finding data similar to the finding data of the target patient, from case data storage means for storing case data of a plurality of patients other than the target patient, the stored case data including the finding data relevant to a brain disease and vital sign data in a treatment period, and setting the searched case data as similar case data; and information output means for acquiring an appropriate range of vital sign data from the similar case data and outputting information regarding a goal of vital sign data of the target patient based on the appropriate range.

An operation method of a medical information providing apparatus of the invention is an operation method of a medical information providing apparatus comprising finding acquisition means, similar case search means, and information output means. The operation method of a medical information providing apparatus comprises: acquiring finding data including disease information regarding a brain disease of a target patient and diseased part information regarding a diseased part where the brain disease has occurred using the finding acquisition means; searching for case data having finding data similar to the finding data of the target patient, from case data storage means for storing case data of a plurality of patients other than the target patient, the stored case data including the finding data relevant to a brain disease and vital sign data in a treatment period, and setting the searched case data as similar case data using the similar case search means; and acquiring an appropriate range of vital sign data from the similar case data and outputting information regarding a goal of vital sign data of the target patient based on the appropriate range using the information output means.

A medical information providing program of the invention causes a computer to function as: finding acquisition means for acquiring finding data including disease information regarding a brain disease of a target patient and diseased part information regarding a diseased part where the brain disease has occurred; similar case search means for searching for case data having finding data similar to the finding data of the target patient, from case data storage means for storing case data of a plurality of patients other than the target patient, the stored case data including the finding data relevant to a brain disease and vital sign data in a treatment period, and setting the searched case data as similar case data; and information output means for acquiring an appropriate range of vital sign data from the similar case data and outputting information regarding a goal of vital sign data of the target patient based on the appropriate range.

The “disease information” indicates any one of brain diseases including cerebral infarction, intracerebral hemorrhage, and subarachnoid hemorrhage. In a case where a plurality of brain diseases occur, information regarding the combination is included.

The “diseased part information” includes at least one or more pieces of information, such as the position, size, shape, and location of a diseased part.

The “appropriate range” refers to a preferable range for treatment of the target patient. For “acquire the appropriate range of the vital sign data from the similar case data”, a range determined with the average value or the representative value of vital sign data of similar case data as a reference may be acquired as the appropriate range. For example, a range having an appropriate width for the average value or the representative value may be acquired as the appropriate range.

The finding acquisition means may perform image analysis processing on a brain image, which is obtained by imaging the target patient, to acquire a position and a range of the diseased part as the diseased part information.

The finding acquisition means may acquire a position and a range of the diseased part as the diseased part information from an interpretation report of a brain image obtained by imaging the target patient.

The finding data may further include treatment information regarding treatment contents for the brain disease.

The “treatment information” refer to information regarding the treatment contents of a brain disease under treatment, such as the presence or absence of surgical treatment, the presence or absence of craniotomy, the presence or absence of intravascular treatment, and the presence or absence of administration of antihypertensive agent.

The finding data may further include anamnesis, medication history, family history, or genetic information.

It is preferable that the case data is case data showing a satisfactory progress in the treatment period, vital sign data of the case data includes a change in blood pressure in the treatment period, and the information output means acquires an appropriate range of blood pressure from similar case data obtained from the case data showing the satisfactory progress.

It is preferable that the case data includes progress information indicating whether a progress in the treatment period is satisfactory or unsatisfactory, vital sign data of the case data includes a change in blood pressure in the treatment period, and the information output means acquires an appropriate range of blood pressure from similar case data obtained from the case data showing the satisfactory progress.

The “case data showing the satisfactory progress in the treatment period” refers to an eventually satisfactory case, and case data that shows an eventually unsatisfactory case even though the progress is satisfactory is not included. In addition, the “case data showing the satisfactory progress in the treatment period” may include an eventually satisfactory cure case even though the progress is once unsatisfactory.

The finding data may include a normal blood pressure.

The “normal blood pressure” is the blood pressure before the brain disease under treatment occurs. For example, the “normal blood pressure” may be a blood pressure measured on a daily basis or a blood pressure measured in a case where a medical examination is performed in the medical department before the onset of the disease.

The similar case search means may comprise similarity calculation means for calculating a similarity between the finding data of the target patient and the finding data in the case data, and case data having the similarity equal to or greater than a threshold value may be set as the similar case data.

The similarity calculation means may express the finding data as a vector obtained by numerically expressing each item forming the disease information and the diseased part information in the finding data, and the similarity may be obtained from a vector of the finding data of the target patient and a vector of the finding data in the case data.

According to the invention, by acquiring the appropriate range of vital sign data from vital sign data in the treatment period of cases similar to finding data including the disease information of the brain disease of the target patient and the diseased part of the brain disease and outputting information regarding the goal of the vital sign data of the target patient, it is possible to check the appropriate vital signs in the treatment period. As a result, it is possible to perform better treatment.

### BRIEF DESCRIPTION OF THE DRAWINGS

FIG. 1 is a diagram showing the schematic configuration of a medical information system.

FIG. 2 is a diagram showing the schematic configuration of a medical information providing apparatus of the invention.

FIG. 3 is a diagram showing an example of a brain image of a patient with a brain disease.

FIG. 4 is a diagram illustrating the similarity of finding data.

FIG. 5 is a flowchart showing the flow of the process of the medical information providing apparatus.

### DETAILED DESCRIPTION

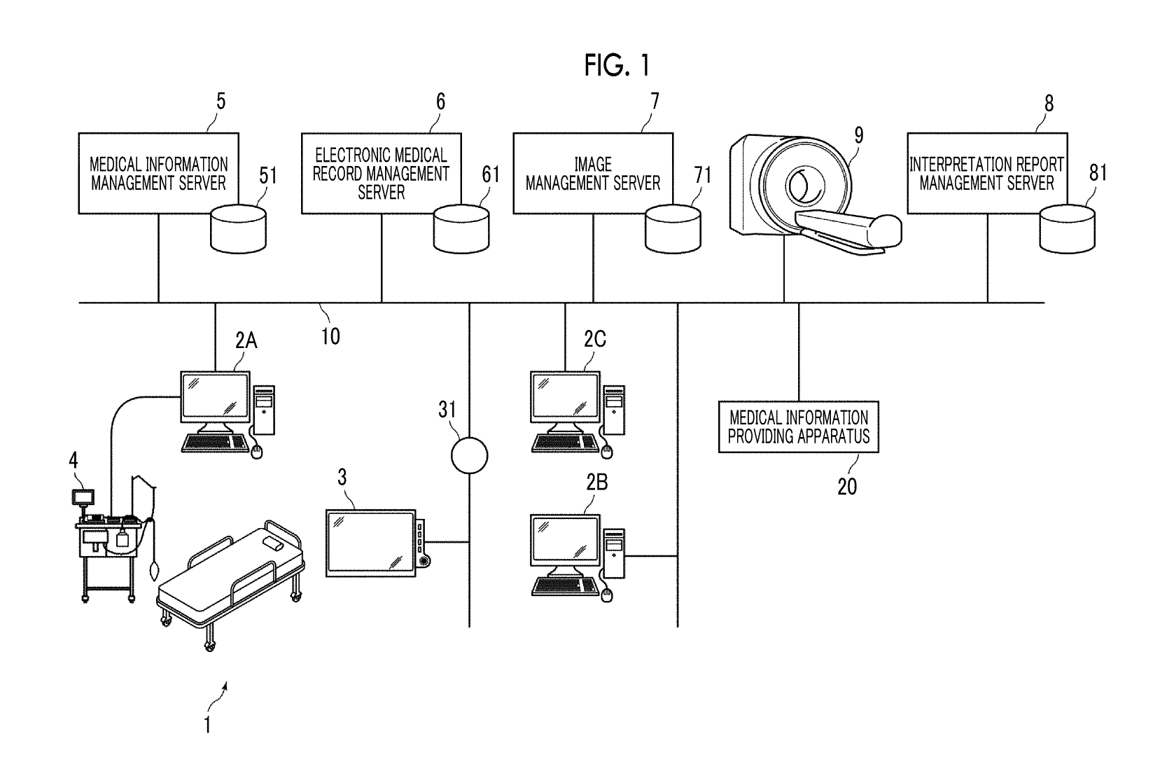

A medical information providing apparatus of the invention will be described with reference to the diagrams. FIG. 1 is a diagram showing the schematic configuration of a medical information system in which a medical information providing apparatus according to an embodiment of the invention is provided.

As shown in FIG. 1, a medical information system 1 has a configuration in which a bedside terminal 2A, a nurse station terminal 2B, a medical department terminal 2C, a biological information monitor 3, a medical information management server 5, an electronic medical record management server 6, an image management server 7, an interpretation report management server 8, and a modality (imaging apparatus) 9 are communicably connected to each other through a network 10 and a medical information providing apparatus 20 of the invention is connected to the network 10.

The biological information monitor 3 includes a measurement unit attached to a patient and a display device that displays vital sign data continuously acquired from the measurement unit, and has a function of monitoring a plurality of vital sign data of the patient. In addition, the biological information monitor 3 has a function of issuing an alarm as necessary. Examination items of the biological information monitor 3 include an electrocardiogram, a heart rate, a respiration rate, a body temperature, an invasive blood pressure, a noninvasive blood pressure, an oxygen saturation (SpO₂), and the like. Vital sign data is acquired from the measurement unit 4, such as an electrocardiogram electrode, a body temperature measurement probe, a cuff, and an SpO₂ measurement probe connected to the biological information monitor 3. The biological information monitor 3 is connected to the network 10 through a biological information monitor interface device 31, and vital sign data is transmitted to the medical information management server 5 so as to be associated with each patient identification (ID). The vital sign data transmitted to the medical information management server 5 is stored in a medical information database 51 for each patient.

The biological information monitor interface device 31 is configured by, for example, a LAN conversion device or a server, and connects the biological information monitor 3 to the medical information management server 5 and each of terminals 2A, 2B, and 2C through the network 10.

The bedside terminal 2A is placed near the patient's bed, is connected to the medical information management server 5 through the network 10, and receives various kinds of information, such as vital sign data of each patient, from the medical information management server 5. The bedside terminal 2A is used to display a progress chart of vital signs of the patient acquired by the biological information monitor 3, input treatment information regarding treatment performed on the patient, such as infusion and transfusion, and the like. The bedside terminal 2A is a computer including a display device, which is a general display, and an input device configured to include a keyboard, a mouse, and the like. In the bedside terminal 2A, not only standard software, such as an operating system, but also application software for viewing the progress chart of the patient or inputting the executed treatment is installed. The bedside terminal 2A is connected to the medical information management server 5 through the network 10. Treatment information is transmitted to the medical information management server 5 so as to be associated with a patient ID and is stored in the medical information database 51 for each patient.

The nurse station terminal 2B is a computer that is placed in a nurse station and is used to check the information of a patient in the intensive care room or instruction information issued for the patient, and includes a display device, which is a general display, and an input device configured to include a keyboard, a mouse, and the like. The nurse station terminal 2B is connected to the medical information management server 5 through the network 10, and is also used to view the progress chart of the patient in the intensive care room. In the nurse station terminal 2B, standard software, such as an operating system, and application software for viewing a progress chart or instruction information issued for the patient are installed.

The medical department terminal 2C is a computer that a doctor or the like of the medical department uses for the input of medical examination information obtained as a result of examining a patient, viewing of an electronic medical record, input of an examination order, and the like. The medical department terminal 2C includes a display device, which is a general display, and an input device configured to include a keyboard, a mouse, and the like. The medical department terminal 2C is connected to the medical information management server 5 through the network 10, and is also used to view the progress chart or instruction information issued for the patient in the intensive care room. In the medical department terminal 2C, standard software, such as an operating system, and application software for inputting medical examination information, viewing an electronic medical record, inputting an examination order, and viewing a progress chart or instruction information issued for the patient are installed. In addition, each process, such as an image viewing request to the image management server 7, display of an image received from the image management server 7, an interpretation report viewing request to the interpretation report management server 8, and display of an interpretation report received from the interpretation report management server 8, is performed by execution of application software.

The electronic medical record management server 6 is a computer including an electronic medical record database 61 in which an electronic medical record is stored, and has an operating system or database management software. The electronic medical record management server 6 is connected to the bedside terminal 2A, the nurse station terminal 2B, and the medical department terminal 2C through the network 10, and information relevant to findings input from each medical department terminal 2C or the like is recorded in the electronic medical record and stored in the electronic medical record database 61.

The medical information management server 5 is a computer including the medical information database 51, and has an operating system or database management software. The medical information management server 5 is connected to the bedside terminal 2A, the nurse station terminal 2B, the medical department terminal 2C, and the electronic medical record management server 6 through the network 10. The medical information management server 5 acquires the patient's vital sign data, treatment information, examination data of various examinations, and the like from each connected server, terminal, or the like based on the patient ID and stores these so as to be associated with the patient ID. In addition, information, such as vital sign data of each patient, treatment information, and examination data of various examinations, is stored in the electronic medical record and stored in the electronic medical record database 61.

A modality 9 includes an apparatus that generates an examination image showing an examination target part of a subject by imaging the examination target part of the subject, adds accessory information specified by the DICOM standard (hereinafter, referred to as a DICOM tag) to the examination image, and outputs the examination image. Specific examples include an MRI apparatus, a CT apparatus, and the like.

The image management server 7 is a computer including an image database 71, and has an operating system or database management software. The image management server 7 includes a large capacity storage in which the image database 71 is formed.

In the image database 71, examination images obtained by imaging a plurality of patients with the modality 9 and accessory information are registered. The accessory information includes, for example, an image ID for identifying each image, a patient ID for identifying a subject, an examination ID for identifying an examination, a unique identification (UID) allocated to each examination image, examination date and examination time at which the examination image is generated, the type of a modality used in an examination for acquiring the examination image, patient information such as patient's name, age, and gender, an examination part (imaging part), imaging conditions (whether or not a contrast medium is used, radiation dose, and the like), and information such as a series number in a case where a plurality of tomographic images are acquired in one examination.

In a case where a viewing request from a workstation for radiologists (not shown) is received through the network 10, the image management server 7 searches for an examination image registered in the image database 71 and transmits the extracted examination image to the workstation for radiologists that is an examination image request source.

The interpretation report management server 8 is a computer including an interpretation report database 81, and has an operating system or database management software. The image management server 7 includes a large capacity storage in which the image database 71 is formed.

In the interpretation report database 81, for example, an interpretation report is registered in which information, such as an image ID for identifying an interpretation target image or a representative image, a radiologist ID for identifying an image diagnostician who performed the interpretation, a lesion name, position information of a lesion, findings, and the certainty of findings, is recorded. In addition, a determination result obtained by the biopsy is recorded in the interpretation report.

The network 10 is a local area network that connects various apparatuses in a hospital to each other. In a case where the workstation for radiologists is installed in another hospital or clinic, the network 10 may be configured to connect local area networks of respective hospitals through the Internet or a dedicated line. In any case, the network 10 is preferably a network capable of realizing high-speed transmission of examination images, such as an optical network.

In the above description, the case has been described in which the examination image and the interpretation report of each patient are stored in the image database 71 and the interpretation report database that are separately provided. However, the interpretation report may be stored in the image database 71 together with the examination image.

The medical information providing apparatus 20 of the invention is configured by a general-purpose computer, and includes known hardware components, such as CPU, a main memory, an auxiliary storage device, an input and output interface, a communication interface, an input device, a display device, and a data bus. A known operating system, a known application program, and the like are installed on the medical information providing apparatus 20. In addition, a medical information providing program of the invention is installed. The medical information providing apparatus 20 has a function of transmitting and receiving data to and from the medical information database 51, the image database 71, and the interpretation report database 81, which are connected to the network 10, through a communication interface. The medical information providing program may be installed from a recording medium, such as a compact disc read only memory (CD-ROM), or may be installed through a network, such as the Internet.

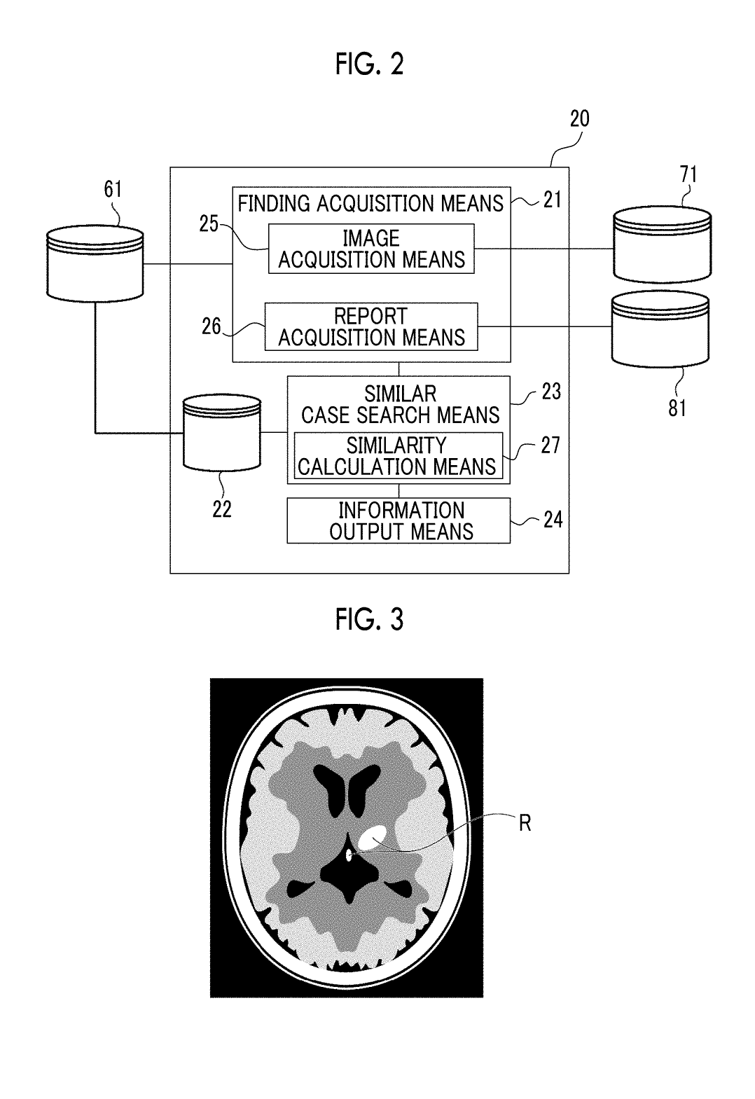

Next, the medical information providing apparatus 20 of the embodiment of the invention will be specifically described with reference to the diagrams. FIG. 2 is a functional block diagram showing the configuration of the medical information providing apparatus 20.

As shown in FIG. 2, the medical information providing apparatus 20 includes finding acquisition means 21, case data storage means 22, similar case search means 23, and information output means 24.

The finding acquisition means 21 acquires finding data of a target patient from the electronic medical record. The finding data includes disease information regarding the brain disease and diseased part information regarding a diseased part where the brain disease has occurred. The disease information is information regarding the type of brain disease, and is configured to have a large classification indicating a largely divided disease condition, such as a stroke, and a small classification indicating a disease condition, such as intracerebral hemorrhage, subarachnoid hemorrhage, and cerebral infarction in the classification. The diseased part information includes information of a lesion position and the volume of a lesion part in each disease condition. For example, the diseased part information is a bleeding position and a bleeding volume in case of bleeding and an infarct position and an infarct volume of an infarction part. The finding data also includes the elapsed time from onset to treatment intervention, chief complaint, level of consciousness, level of paralysis, paralyzed part, and the like as information indicating the onset condition.

Alternatively, image acquisition means 25 and report acquisition means 26 may be provided in the finding acquisition means 21 so that information regarding the diseased part is acquired from the brain image or the interpretation report.

The image acquisition means 25 acquires a brain image, which is obtained by imaging the target patient with reference to the DICOM tag, from the image database 71, and the finding acquisition means 21 acquires the position and the range (including the volume) of the diseased part as diseased part information by performing image analysis processing on the acquired image. FIG. 3 is an example of a CT image of a target patient. By performing image processing, a bleeding region R (white region in the vicinity of the center of the image) is detected from the thalamus. From the number of pixels determined to be the bleeding region R and the pixel interval, the volume of the bleeding region is calculated. For example, in the example shown in FIG. 3, the bleeding region R is 35 ml.

In the case of brain diseases, not only a CT image but also an MRI image is used for diagnosis in many cases. In particular, an MRI image is generally used for diagnosis of cerebral infarction. In a case where there is an MRI image of the same target patient, the image acquisition means 25 can also detect the position and the range of the diseased part of cerebral infarction by performing image processing on the MRI image.

The report acquisition means 26 acquires an interpretation report of a brain image, which is obtained by imaging the target patient, from the interpretation report database 81, and the finding acquisition means 21 acquires the position and the range of the diseased part as diseased part information from the acquired interpretation report. By analyzing the character string of the interpretation report associated with each acquired image, information regarding the diseased part that is recorded in the interpretation report is extracted. Specifically, sentences in the interpretation report are segmented into words by performing natural language processing, and information regarding the diseased part is acquired by analyzing the sequence of words. As necessary, terms such as a part name are registered in advance, and contents described in the report are extracted from the character string matching the terms. In addition, information regarding the bleeding volume or the lesion volume is acquired from the combination of a character string indicating a numerical value and a character string indicating the unit of a numerical value (for example, “ml”). For terms, a medical term dictionary may be prepared in advance. A plurality of terms referring to the same disease, the same lesion, or the same part may be registered in a dictionary, and terms recorded in the interpretation report that refer to the same disease or the same part may be regarded as the same term. As in a fuzzy search, even for a term that does not exactly match the term in the dictionary under a certain rule, the matching of the term may be determined using the closest term in the dictionary.

The finding data may include treatment information regarding the contents of treatment performed on the brain disease. The treatment contents are the contents of treatment performed during the treatment period, such as the presence or absence of surgical treatment, the presence or absence of craniotomy, the presence or absence of intravascular treatment, and the presence or absence of administration of antihypertensive agent. The treatment information can be acquired from the electronic medical record.

In the case of brain diseases, not only the current state of the disease, such as age, gender, or anamnesis, but also other factors often have a great influence on the selection of treatment policy. Therefore, the finding data may further include patient information, such as age, gender, height, and weight, and information, such as anamnesis, medication history, family history, or genetic information.

In control of blood pressure, lowering the blood pressure of a person who originally has high blood pressure has a risk that the brain will become ischemic. Therefore, the finding data may include normal blood pressure. Examples of the normal blood pressure include a blood pressure measured on a daily basis before the onset of a brain disease currently being treated and a blood pressure measured in a case where a medical examination is performed in the medical department before the onset of the disease. The normal blood pressure may be a value obtained by recording information heard from a patient or his or her family in the electronic medical record at the time of medical examination or a value obtained by recording a measurement result in the medical department before the onset of the disease in the electronic medical record.

The case data storage means 22 may be an auxiliary storage device, such as a hard disk provided in the medical information providing apparatus 20, or may be an external storage device connected to the network 10, such as a network attached storage (NAS).

The case data is data of a plurality of patients other than the target patient. The case data includes finding data relevant to brain disease or diseases and vital sign data in the treatment period from the start of treatment of each patient to discharge. The vital sign data includes changes in blood pressure (invasive blood pressure, noninvasive blood pressure), heart rate, respiration rate, and body temperature measured during the treatment period. The vital sign data is obtained from the biological information monitor 3 attached to each patient during the treatment period and is recorded as medical examination information, and the vital sign data is stored so as to be associated with the electronic medical record.

It is preferable that the case data is data relevant to cases showing the satisfactory progress in the treatment period. Therefore, electronic medical records and vital sign data of patients showing the satisfactory progress, among brain disease patients, from the electronic medical record database 61 are stored as case data in the case data storage means 22 in advance. The case data may be data in which progress information indicating whether the progress in the treatment period is satisfactory or unsatisfactory is added to data configured to include an electronic medical record and vital sign data of a patient with a brain disease. The case data may include items corresponding to finding data and vital sign data, or may be the electronic medical record itself of a patient with a brain disease that shows the satisfactory progress.

The similar case search means 23 includes similarity calculation means 27, and calculates a similarity, searches for case data having finding data similar to the finding data of the target patient from the case data storage means 22, and sets the case data as similar case data.

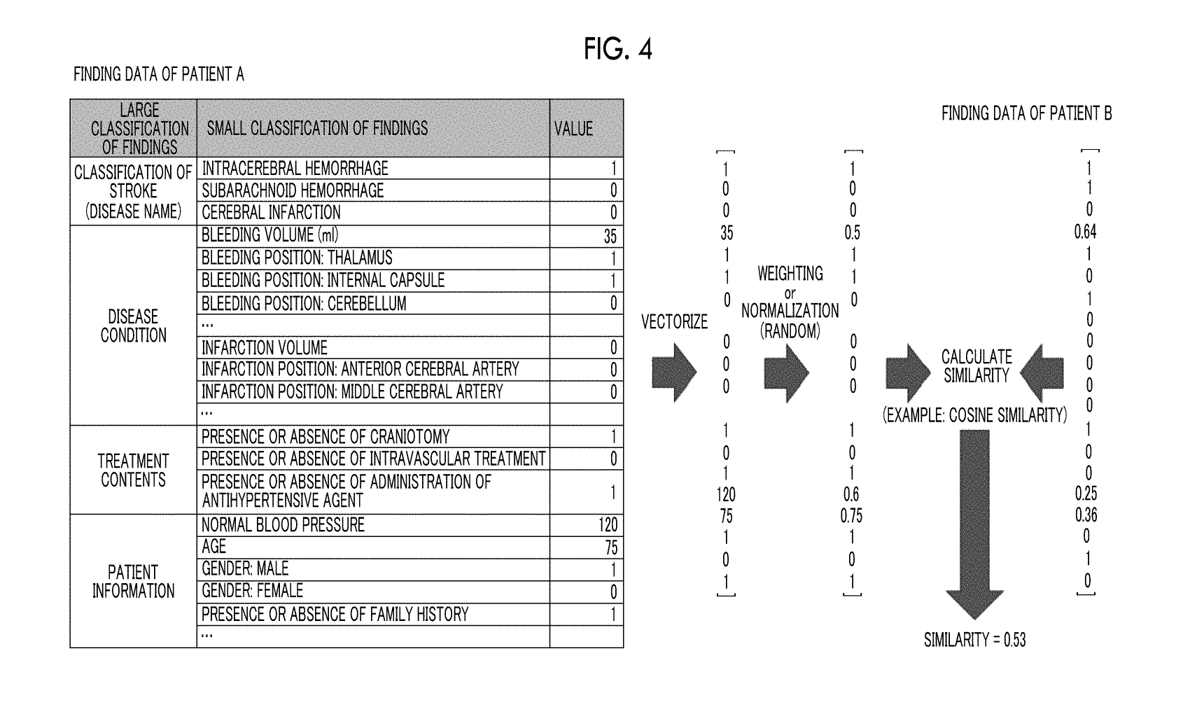

The similarity calculation means 27 calculates a similarity between the finding data of the target patient and the finding data in the case data. Each item forming the disease information of the finding data and the diseased part information is numerically expressed and represented by a vector, and the similarity is calculated from the vector of the finding data of the target patient and the finding data in the case data. FIG. 4 shows an example in which finding data is vectorized. In the example shown in FIG. 4, for the disease information in the finding data, a vector component of an item corresponding to the disease condition of the small classification is set to “1”, and an item not corresponding to the disease condition of the small classification is set to “0”. For the bleeding position, a vector component of an item corresponding to the position (for example, “thalamus”, “internal capsule”, or “cerebellum”) of the small classification is set to “1”, and an item not corresponding to the position of the small classification is set to “0”. Similarly, also for the infarct position, a vector component of an item corresponding to the position of the small classification is set to “1”, and an item not corresponding to the position of the small classification is set to “0”. For items expressed as numerical values, such as the bleeding volume or the normal blood pressure, numerical values are used as components of vectors. In addition, vector components of items having a wide numerical range, such as the bleeding volume or the normal blood pressure, are normalized. For example, a numerical value between 0 and 1 is set with the maximum value as a reference. Alternatively, a magnification with respect to the average value with the average value as a reference may be used. Normalization may be performed so that the numerical value of each item becomes a desirable value for an operation, such as comparison or calculation, by changing the numerical value of each item based on a certain rule as described above.

Specifically, the similarity is obtained from the inner product of two vectors. FIG. 4 shows an example of calculating a cosine similarity for which the similarity between a target patient (patient A) and a patient B of case data is obtained from the inner product. In this example, the similarity is 0.53. Although the case where the vector component is normalized has been described above, the similarity may also be calculated by multiplying a weighting coefficient according to the degree of contribution of the similarity of each component without performing normalization.

The similar case search means 23 specifies case data having a similarity equal to or greater than a threshold value, among pieces of case data, as similar case data. Alternatively, a predetermined number of pieces of case data from case data having the highest similarity value, among pieces of case data, may be specified as similar case data.

The information output means 24 acquires the appropriate range of the vital sign data from the similar case data, and outputs information regarding the goal of the vital sign data of the target patient based on the appropriate range to the display device, such as a CRT. The output of information regarding the goal of the vital sign data includes display of a target value of vital data, display of an appropriate range of the value of vital data, display of both appropriate ranges for comparison between the appropriate range of the patient and the standard normal range, display as to which one is better between a value higher than the standard value and a value lower than the standard value (for example, “set blood pressure to be higher than normal blood pressure” or “set blood pressure to be lower than normal blood pressure”), and the like. In addition, without being limited to the display device, any means that can be recognized by the user by changing the color with a lamp or the like may be used. In particular, it is desirable to make sure that a range that is different from the standard normal range, which is generally indicated by treatment guidelines, is within the appropriate range for the target patient.

Specifically, the value of target vital data is calculated, and transmitted to the bedside terminal 2A, the nurse station terminal 2B, and the medical department terminal 2C through the network 10, and displayed on the screen of each of the terminals 2A, 2B, and 2C. For example, assuming that the value of vital sign data of similar case data obtained from case data showing the satisfactory progress is within the normal range of the vital sign, an average value or a representative value of vital sign data of similar cases may be displayed on the screen of each of the terminals 2A, 2B, and 2C. In addition, a range having an appropriate width for the average value or the representative value may be set as an appropriate range. In particular, control of blood pressure is an important item for treatment for patients with brain diseases. Therefore, an average value or a representative value of systolic blood pressure values and diastolic blood pressure values of case data having pieces of similar finding data may be displayed as a target value. The display of such an average value or a representative value indicates that it is preferable to control the vital sign in a range having a certain width from the average value or the representative value, and it is preferable that health care professionals, such as a doctor and a nurse, can recognize as an appropriate range. Alternatively, a value obtained by giving a width to the average value or the representative value may be displayed as a target value.

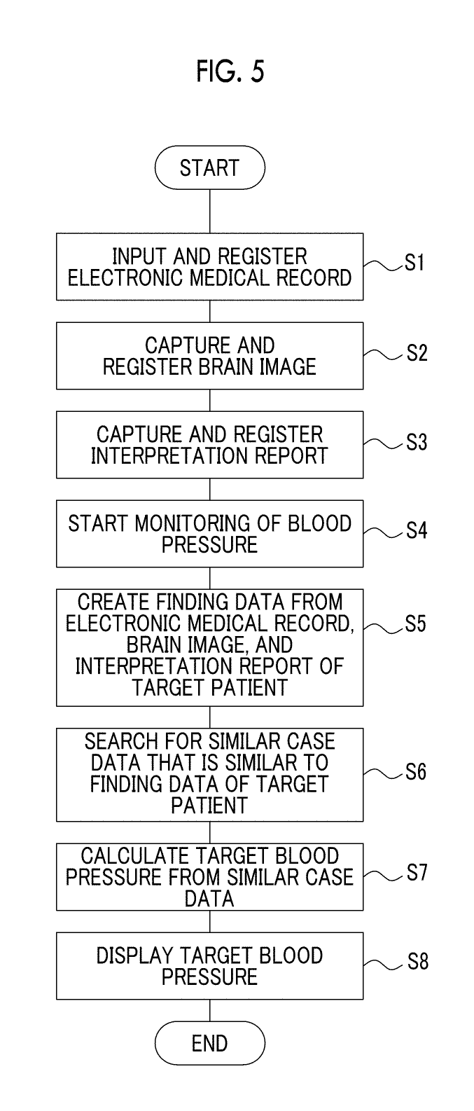

Hereinafter, with reference to the flowchart shown in FIG. 5, a method of providing a goal of an appropriate vital sign of a target patient with a brain disease will be specifically described with blood pressure as an example.

First, in a case where the target patient visits a hospital, interview, image examination, and the like are performed. Information obtained by the interview is recorded in the electronic medical record as information regarding findings by the doctor, and the electronic medical record is stored in the electronic medical record database 61 (S1). The doctor inputs an examination order for image examination according to the symptoms of the target patient. Imaging is performed by using the modality 9 according to the examination order, and the captured brain image is stored in the image database 71 (S2). Then, image interpretation is performed by the radiologist, and an interpretation report is stored (and registered) in the interpretation report database 81 (S3).

According to the result of the diagnosis, treatment is started, and surgical treatment and administration of antihypertensive agent are performed. In addition, the biological information monitor 3 is attached to the target patient, and vital signs, such as blood pressure, are monitored during the treatment period (S4).

On the other hand, in the medical information providing apparatus 20, the finding acquisition means 21 collects an electronic medical record, a brain image, and an interpretation report corresponding to the patient ID of the target patient from the respective databases 61, 71, and 81 to acquire the finding data of the target patient (S5). The similar case search means 23 searches for case data having finding data with high similarity to the finding data of the target patient as similar case data (S6).

The information output means 24 acquires the average value of “systolic blood pressure value/diastolic blood pressure value” from the blood pressure of the similar case data as target blood pressure (S7). The acquired target blood pressure is displayed on the screen of each of the terminals 2A, 2B, and 2C (S8).

In the present embodiment, the case has been described in which the case data storage means 22 is provided in the medical information providing apparatus 20. However, the case data storage means 22 may be provided outside the medical information providing apparatus 20 so as to be placed in servers operated by other hospitals or other institutions or in a cloud.

As described in detail above, by obtaining the target values of vital signs from case data having findings similar to those of a patient with a brain disease, better treatment can be performed by showing an appropriate vital sign goal even for a target patient who needs to be managed differently from vital signs that are generally said to be in the appropriate range.

For case data, it is preferable to search for similar cases from data of cases showing the satisfactory progress in the treatment period. However, similar cases may be searched for from data including data of cases showing the unsatisfactory progress in the treatment period. Even in a case where data of poor cases is included, in a case where the cases are similar cases, it is possible to obtain an appropriate vital sign goal since completely different cases are excluded.

1. A medical information providing apparatus, comprising:
finding acquisition means for acquiring finding data including disease information regarding a brain disease of a target patient and diseased part information regarding a diseased part where the brain disease has occurred;
similar case search means for searching for case data having finding data similar to the finding data of the target patient, from case data storage means for storing case data of a plurality of patients other than the target patient, the stored case data including the finding data relevant to a brain disease and vital sign data in a treatment period, and setting the searched case data as similar case data; and
information output means for acquiring an appropriate range of vital sign data from the similar case data and outputting information regarding a goal of vital sign data of the target patient based on the appropriate range.

2. The medical information providing apparatus according to claim 1,
wherein the finding acquisition means performs image analysis processing on a brain image, which is obtained by imaging the target patient, to acquire a position and a range of the diseased part as the diseased part information.

3. The medical information providing apparatus according to claim 1,
wherein the finding acquisition means acquires a position and a range of the diseased part as the diseased part information from an interpretation report of a brain image obtained by imaging the target patient.

4. The medical information providing apparatus according to claim 1,
wherein the finding data further includes treatment information regarding treatment contents for the brain disease.

5. The medical information providing apparatus according to claim 1,
wherein the finding data further includes anamnesis, medication history, family history, or genetic information.

6. The medical information providing apparatus according to claim 1,
wherein the case data is case data showing a satisfactory progress in the treatment period,
vital sign data of the case data includes a change in blood pressure in the treatment period, and
the information output means acquires an appropriate range of blood pressure from similar case data obtained from the case data showing the satisfactory progress.

7. The medical information providing apparatus according to claim 1,
wherein the case data includes progress information indicating whether a progress in the treatment period is satisfactory or unsatisfactory,
vital sign data of the case data includes a change in blood pressure in the treatment period, and
the information output means acquires an appropriate range of blood pressure from similar case data obtained from the case data showing the satisfactory progress.

8. The medical information providing apparatus according to claim 6,
wherein the finding data includes a normal blood pressure.

9. The medical information providing apparatus according to claim 7,
wherein the finding data includes a normal blood pressure.

10. The medical information providing apparatus according to claim 1,
wherein the similar case search means comprises similarity calculation means for calculating a similarity between the finding data of the target patient and the finding data in the case data, and
case data having the similarity equal to or greater than a threshold value is set as the similar case data.

11. The medical information providing apparatus according to claim 10,
wherein the similarity calculation means expresses the finding data as a vector obtained by numerically expressing each item forming the disease information and the diseased part information in the finding data, and
the similarity is obtained from a vector of the finding data of the target patient and a vector of the finding data in the case data.

12. An operation method of a medical information providing apparatus comprising finding acquisition means, similar case search means, and information output means, the method comprising:
acquiring finding data including disease information regarding a brain disease of a target patient and diseased part information regarding a diseased part where the brain disease has occurred using the finding acquisition means;
searching for case data having finding data similar to the finding data of the target patient, from case data storage means for storing case data of a plurality of patients other than the target patient, the stored case data including the finding data relevant to a brain disease and vital sign data in a treatment period, and setting the searched case data as similar case data using the similar case search means; and
acquiring an appropriate range of vital sign data from the similar case data and outputting information regarding a goal of vital sign data of the target patient based on the appropriate range using the information output means.

13. A non-transitory computer-readable recording medium storing therein a medical information providing program causing a computer to function as:
finding acquisition means for acquiring finding data including disease information regarding a brain disease of a target patient and diseased part information regarding a diseased part where the brain disease has occurred;
similar case search means for searching for case data having finding data similar to the finding data of the target patient, from case data storage means for storing case data of a plurality of patients other than the target patient, the stored case data including the finding data relevant to a brain disease and vital sign data in a treatment period, and setting the searched case data as similar case data; and
information output means for acquiring an appropriate range of vital sign data from the similar case data and outputting information regarding a goal of vital sign data of the target patient based on the appropriate range.
